(19) United States
(12) Patent Application Publication
(10) Pub. No.: US 2015/0246032 A1
(43) Pub. Date: Sep. 3, 2015

(54) PHARMACEUTICAL COMPOSITION COMPRISING REBAMIPIDE

(71) Applicant: OTSUKA PHARMACEUTICAL CO., LTD., Tokyo (JP)

(72) Inventors: Yuji Sugahara, Osaka (JP); Tomohiro Kaiden, Osaka (JP)

(73) Assignee: OTSUKA PHARMACEUTICAL CO. LTD., Tokyo (JP)

(21) Appl. No.: 14/431,542

(22) PCT Filed: Sep. 27, 2013
(86) PCT No.: PCT/JP2013/077011

§ 371 Date: Mar. 26, 2015

Related U.S. Application Data
(60) Provisional application No. 61/707,386, filed on Sep. 28, 2012.

(51) Int. Cl. A61K 31/4704; A61K 47/32; A61K 9/08; A61K 47/24; A61K 47/10; A61K 9/00; A61K 47/02; A61K 47/26

(52) U.S. Cl. CPC A61K 31/4704; A61K 47/02; A61K 47/32; A61K 47/26; A61K 47/24; A61K 47/10; A61K 9/0048; A61K 9/08

(57) ABSTRACT

The present invention relates to a rebamipide-containing ophthalmic composition which achieves a superior re-dispersibility and transparency by comprising various additives as well as a zinc compound as a preservative (which supplements the antiseptic-effect) in the amount which does not produce an adverse effect.

### TECHNICAL FIELD

The present invention relates to an ophthalmic pharmaceutical composition of rebamipide.

### BACKGROUND ART

Rebamipide [chemical name: (±)-2-(4-chlorobenzoyl-amino)-3-[2-quinolon-4-yl]propionic acid] is known to be useful as an antiulcer drug.

In addition, rebamipide increases the goblet cell density, mucous secretion and tear fluid in the eye, and has been already known as an agent for treating dry eye, i.e., dry eye syndrome (Patent Reference 1).

Although rebamipide is soluble in an alkaline aqueous solution, the solubility of rebamipide in a neutral solution is quite poor. Also, an eye drop having a high pH is not suitable for the treatment of an injury in keratoconjunctiva such as dry eye. Furthermore, the development of a formulation containing rebamipide as an aqueous solution is thought to be difficult, since crystalline rebamipide may be precipitated even in case of an alkaline solution thereof.

Patent Reference 1 discloses a neutral aqueous suspension of rebamipide. However, the aqueous suspension should be shaken well for re-dispersion, since a precipitate layer may be formed upon standing for a long period. Also, such formulations might have some problems such as visual defect caused by blurred vision and making some spots upon spilling on the cloths, because the formulation is a white ophthalmic suspension.

Patent Reference 2 discloses an aqueous suspension comprising rebamipide wherein rebamipide can be stably dispersed as fine particles and the fine particles are not re-agglutinated. Although the suspensibility of the rebamipide-containing aqueous suspension of Patent Reference 2 is improved compared with that of the formulation of Patent Reference 1, the formation of Patent Reference 2 is unavoidable to form a precipitate layer upon standing for a long period and a vigorous shaking will be needed for the re-dispersion, since the formulation of Patent Reference 2 is also a white ophthalmic suspension in which rebamipide is not completely dissolved. Furthermore, the problems such as visual defect caused by blurred vision and making some spots upon spilling on the cloths remain unresolved even in Patent Reference 2.

Patent Reference 3 discloses an aqueous suspension of crystalline rebamipide comprising a mixture of one or more compounds selected from water-soluble polymers and surfactants, an acidic solution, and a solution of water-soluble salt of rebamipide as a neutral rebamipide-containing pharmaceutical composition with an improved transparency which need not to be re-dispersed and does not cause damages to the keratoconjunctiva of patients suffering from dry eye.

However, the aqueous suspension of crystalline rebamipide of Patent Reference 3 has a problem of high production cost, since expensive equipments such as a high-pressure homogenizer, a colloidmill and sonicator are required during its formulation processes, and the manufacturing process is cumbersome, complicated and long-haul.

Patent Reference 4 discloses a preservative-free pharmaceutical composition comprising rebamipide as an eye drop having an improved re-dispersibility, transparency and storage stability. The eye drop of Patent Reference 4 is free from an inorganic cation in order to avoid the addition of a preservative which might be toxic against patients suffering from an injury of cornea such as dry eye. However, preventing microbial contamination without a preservative is not so enough from the view of safety, and thus, the development of an effective preservative method having no harmful effects has been desired.

Patent Reference 5 and Patent Reference 6 disclose that compounds such as zinc chloride are effective for preventing microbial contamination in an eye drop. However, the concentration of such zinc compounds for preventing microbial contamination is 0.001% (W/V) to 0.0001% (W/V). These concentrations cannot be recognized as a sufficiently low concentration, considering the adverse effects and costs caused by adding zinc compounds.

### PATENT REFERENCE

[Patent Reference 1] WO 1997/013515

[Patent Reference 2] WO 2008/050896

[Patent Reference 3] WO 2006/052018

[Patent Reference 4] WO 2009/154304

[Patent Reference 5] JP 2010-504990 T

[Patent Reference 6] JP 2010-504358 T

### SUMMARY OF INVENTION

### Problems to be Solved by the Invention

The present invention relates to rebamipide-containing eye drop and provides a pharmaceutical composition which has a superior re-dispersibility and transparency and contains a preservative (an agent for supplementing preservative effectiveness) which is not generally suitable for the treatment of a patient suffering from injury in cornea such as dry eye, in a small amount that is expected to produce a sufficient antiseptic-effect in spite of such small amount, without causing undesirable effects.

### Means to Solve the Problems

The present inventors have intensively studied and found that a rebamipide-containing eye drop having a superior re-dispersibility, transparency and a sufficient antiseptic-effect can be prepared by adding a trace amount of a zinc compound as a preservative (which supplements the antiseptic-effect) and various particular additives. Based upon the new findings, the present invention has been completed.

The present invention provides pharmaceutical compositions and use thereof as shown in the following [Term 1] to [Term 14].

[Term 1] A pharmaceutical composition comprising (1) rebamipide, (2) a solubilizing agent, (3) an amino sugar, (4) a buffer and (5) a zinc compound.

[Term 2] The pharmaceutical composition of Term which is an aqueous liquid.

[Term 3] The pharmaceutical composition of Term 1 or 2 wherein the concentration of zinc in the zinc compound is 0.000001 to 0.00005% (w/v).

[Term 4] The pharmaceutical composition of Term 1 or 2 wherein the concentration of zinc in the zinc compound is 0.000001 to 0.000004% (w/v).

[Term 5] The pharmaceutical composition of any one of Terms 1 to 4 wherein the concentrations of rebamipide, the solubilizing agent, the amino sugar, and the buffer are 1 to 3% (w/v), 2 to 4% (w/v), 1 to 6% (w/v), and 0.05 to 2% (w/v), respectively.

[Term 6] The pharmaceutical composition of any one of Terms 1 to 5 wherein the zinc compound is zinc chloride and/or zinc sulfate.

[Term 7] The pharmaceutical composition of any one of Terms 1 to 6 wherein the solubilizing agent is polyvinylpyrrolidone.

[Term 8] The pharmaceutical composition of any one of Terms 1 to 7 wherein the amino sugar is meglumine.

[Term 9] The pharmaceutical composition of any one of Terms 1 to 8 wherein the buffer is boric acid and/or phosphoric acid.

[Term 10] The pharmaceutical composition of any one of Terms 1 to 9 further comprising an isotonic agent.

[Term 11] The pharmaceutical composition of Term 10 wherein the isotonic agent is glycerin.

[Term 12] The pharmaceutical composition of any one of Terms 1 to 11 wherein pH is in a range of 7 to 9.

[Term 13] The pharmaceutical composition of any one of Terms 1 to 12 wherein the pharmaceutical composition is an ophthalmic pharmaceutical composition.

[Term 14] Use of the pharmaceutical composition of any one of Terms 1 to 13 for treating dry eye by topically administering to eyes.

### Effect of the Invention

The present rebamipide-containing pharmaceutical composition acquire a superior re-dispersibility, transparency and a sufficient antiseptic-effect by comprising a zinc compound as a preservative (which supplements the antiseptic-effect) in a trace amount which does not produce adverse effects.

### DESCRIPTION OF EMBODIMENTS

The pharmaceutical composition of the present invention is preferably formulated as an aqueous liquid, and more preferably used as an ophthalmic pharmaceutical composition.

The concentration of rebamipide in the pharmaceutical composition of the present invention is in a range of about 0.1 to about 5% (w/v), preferably about 0.5 to about 3% (w/v), and more preferably about 1 to about 3% (w/v). As used herein, 1% (w/v) means that 100 mL of the solution contains 1 g of the ingredient.

Examples of the solubilizing agents used herein include polymers such as polyvinylpyrrolidone, macrogol (polyethylene glycol), polyvinyl alcohol and hydroxypropylmethylcellulose; surfactants such as polysorbate, polyoxyethylene hydrogenated castor oil, and polyoxyethylene-polyoxypropylene; polyhydric alcohols such as propylene glycol; organic acids such as benzoic acid and sorbic acid; amino acids such as alginic acid, histidine, glycine and lysine; and xanthine derivatives such as caffeine. The preferred solubilizing agent is polyvinylpyrrolidone, macrogol, polyvinyl alcohol, benzoic acid, sorbic acid and alginic acid, and polyvinylpyrrolidone is especially preferable. These solubilizing agents can be used alone or as a combination of any two or more.

The molecular weight of the polyvinylpyrrolidone used herein is preferably 200000 or less, and more preferably 40000 or less. The preferred polyvinylpyrrolidones includes, but is not limited to, polyvinylpyrrolidone (PVP, purchased from BASF, Kollidon 25): PVP (K-25), polyvinylpyrrolidone (PVP, purchased from BASF, Kollidon 17PF): PVP (K-17PF). The ratio of rebamipide versus polyvinylpyrrolidone is preferably 20:1 to 1:20, more preferably 4:1 to 1:6, and even more preferably 1:1 to 1:2.

When a solubilizing agent is added, the concentration of the solubilizing agent is generally in a range of about 0.01 to about 15 (w/v) %, preferably about 0.1 to about 10 (w/v) %, more preferably about 0.5 to about 6 (w/v) %, and even more preferably about 1 to about 5 (w/v) % or about 2 to about 4 (w/v) %.

The zinc compounds used herein are, for example, zinc chloride and zinc sulfate, and zinc chloride is preferred. These zinc compounds can be used alone or as a combination of any two or more.

The amount of zinc in the zinc compound in the present pharmaceutical composition is about 0.0000005 to about 0.000025 times, preferably about 0.0000005 to about 0.00001 times, more preferably 0.0000005 to about 0.000005 times, and even more preferably about 0.0000005 to about 0.000002 times per the weight of rebamipide.

The concentration of zinc in the zinc compounds in the present pharmaceutical composition is preferably in a range of about 0.000001 to about 0.00005% (w/v), more preferably about 0.000001 to about 0.00002 (w/v), even more preferably about 0.000001 to about 0.00001% (w/v), and the most preferably about 0.000001 to about 0.000004% (w/v). The range of about 0.000002 to about 0.0000035% (w/v) or about 0.000002 to about 0.0000033% (w/v) is also preferred from the viewpoint of the solubility of the zinc compounds under various storage conditions of the pharmaceutical composition.

Examples of amino sugars used herein include meglumine (i.e., N-methyl-D-glucamine), D-glucosamine, D-galactosamine, D-mannosamine, mycosamine, kanosamine, neosamine C, N-methyl-L-glucosamine, mycaminose, muramic acid, and streptamine.

Amino sugars selected from the group consisting of meglumine, D-glucosamine, D-galactosamine, D-mannosamine, mycosamine, kanosamine, neosamine C, N-methyl-L-glucosamine, mycaminose, muramic acid and streptamine are preferred, and the most preferred is meglumine. These amino sugars can be used alone or as a combination of any two or more.

The concentration of the amino sugar in the present pharmaceutical composition may be, for example, in a range of about 0.1 to about 15% (w/v), preferably about 0.5 to about 10% (w/v), more preferably about 1 to about 8 (w/v) or about 1 to about 6% (w/v).

Examples of buffers used herein include boric acid, phosphoric acid, amino acid, and organic acids, and boric acid and phosphoric acid are preferable. These buffers can be used alone or as a combination of any two or more.

The concentration of the buffer in the present pharmaceutical composition is, for example, in a range of about 0.01 to about 4% (w/v), preferably about 0.03 to about 3% (w/v), and more preferably about 0.05 to about 2% (w/v).

If necessary, an isotonic agent may be added to the present pharmaceutical composition in order to ensure that the composition is isotonic with lacrimal fluid. A conventional isotonic agent for an ophthalmic solution such as mannitol, glycerin, propylene glycol, polyethylene glycol, maltose, sucrose, sorbitol, and glucose can be used for the pharmaceutical composition of the present invention, and glycerin and sucrose are preferred. These isotonic agents can be used alone or as a combination of any two or more.

The concentration of the isotonic agent in the present pharmaceutical composition is, for example, in a range of about 0.1 to about 5% (w/v), preferably about 0.2 to about 3% (w/v), more preferably about 0.5 to about 2% (w/v).

A pH adjuster can be used in the present pharmaceutical composition, if necessary. Examples of the pH adjusters include a conventional acid such as hydrochloric acid, lactic acid, acetic acid, sulfuric acid, nitric acid, carbonic acid, phosphoric acid, and citric acid. Among them, hydrochloric acid is preferred. These pH adjusters can be used alone or as a combination of any two or more.

The pH of the aqueous liquid formulation of the present invention is about 3 to about 9, preferably about 7 to about 9.

A conventional preservative besides a zinc compound (e.g., tertiary ammonium salts such as benzalkonium chloride and benzethonium chloride; p-hydroxybenzoate esters such as chlorhexidine gluconate, methyl-p-hydroxybenzoate and propyl-p-hydroxybenzoate; and alcohols such as chlorobutanol and benzyl alcohol) and/or a stabilizer (e.g., inorganic cation-free ascorbic acid, and tocopherol) can be added to the present pharmaceutical composition, if desired.

The present pharmaceutical composition may contain a conventional ophthalmic ingredient besides rebamipide such as an antihistamine, antiallergic drug, vitamins, an anti-inflammatory drug and an ingredient for relieving redness of the eye, and/or a conventional ophthalmic additive such as a thickener, a chelating agent, a suspending agent, an emulsifying agent, and an antioxidant, if necessary.

### EXAMPLE

Hereinafter, the present invention is illustrated by the following examples, but should not be construed to be limited thereto.

### Example 1

| rebamipide | 2 | g |
|---|---|---|
| polyvinylpyrrolidone K17 | 3 | g |
| boric acid | 1.5 | g |
| meglumine | 5.7 | g |
| glycerin | 0.819 | g |
| hydrochloric acid | q.s. (pH 8.5) | |
| zinc chloride | 2.09 μg (as 1 μg of zinc) | |
| purified water | q.s. | |
| Total | 100 | mL |

According to each amount defined in the above table, to an appropriate volume of purified water with stirring were added rebamipide, polyvinylpyrrolidone K17, boric acid, meglumine, glycerin, and zinc chloride. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 2

A pharmaceutical composition which was a colorless to light yellow clear solution was prepared according to the procedure described in Example 1 except that 10.4 μg of zinc chloride (as 5 μg of zinc) was employed.

### Example 3

A pharmaceutical composition which was a colorless to light yellow clear solution was prepared according to the procedure described in Example 1 except that 20.9 μg of zinc chloride (as 10 μg of zinc) was employed.

### Example 4

A pharmaceutical composition which was a colorless to light yellow clear solution was prepared according to the procedure described in Example 1 except that 41.7 μg of zinc chloride (as 20 μg of zinc) was employed.

### Example 5

A pharmaceutical composition which was a colorless to light yellow clear solution was prepared according to the procedure described in Example 1 except that 104.3 μg of zinc chloride (as 50 μg of zinc) was employed.

### Example 6

| rebamipide | 2 | g |
|---|---|---|
| polyvinylpyrrolidone K17 | 3 | g |
| boric acid | 1.5 | g |
| meglumine | 5.7 | g |
| glycerin | 0.819 | g |
| hydrochloric acid | q.s. (pH 8.5) | |
| zinc sulfate 7-hydrate | 4.4 μg (as 1 μg of zinc) | |
| purified water | q.s. | |
| Total | 100 | mL |

According to each amount defined in the above table, to an appropriate volume of purified water with stirring were added rebamipide, polyvinylpyrrolidone K17, boric acid, meglumine, glycerin, and zinc sulfate 7-hydrate. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 7

A pharmaceutical composition which was a colorless to light yellow clear solution was prepared according to the procedure described in Example 6 except that 22 μg of zinc sulfate 7-hydrate (as 5 μg of zinc) was employed.

### Example 8

A pharmaceutical composition which was, a colorless to light yellow clear solution was prepared according to the procedure described in Example 6 except that 44 μg of zinc sulfate 7-hydrate (as 10 μg of zinc) was employed.

### Example 9

A pharmaceutical composition which was a colorless to light yellow clear solution was prepared according to the procedure described in Example 6 except that 88 μg of zinc sulfate 7-hydrate (as 20 μg of zinc) was employed.

### Example 10

A pharmaceutical′ composition which was a colorless to light yellow clear solution was prepared according to the procedure described in Example 6 except that 220 μg of zinc sulfate 7-hydrate (as 50 μg of zinc) was employed.

### Comparative Example 1

| rebamipide | 2 | g |
|---|---|---|
| polyvinylpyrrolidone K17 | 3 | g |
| boric acid | 1.5 | g |
| meglumine | 5.7 | g |
| glycerin | 0.819 | g |
| hydrochloric acid | q.s. (pH 8.5) | |
| purified water | q.s. | |
| Total | 100 | mL |

According to each amount defined in the above table, to an appropriate volume of purified water with stirring were added rebamipide, polyvinylpyrrolidone K17, boric acid, meglumine, and glycerin. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 11

| rebamipide | 2 | g |
|---|---|---|
| boric acid | 1.5 | g |
| meglumine | 5.7 | g |
| glycerin | 0.819 | g |
| hydrochloric acid | q.s. (pH 8.5) | |
| zinc chloride | 41.7 μg (as 20 μg of zinc) | |
| purified water | q.s. | |
| Total | 100 | mL |

According to each amount defined in the above table, to an appropriate volume of purified water with stirring were added rebamipide, boric acid, meglumine, glycerin, and zinc chloride. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 12

A pharmaceutical composition which was a colorless to light yellow clear solution was prepared according to the procedure described in Example 11 except that 104.3 μg of zinc chloride (as 50 μg of zinc) was employed.

### Comparative Example 2

| rebamipide | 2 | g |
|---|---|---|
| boric acid | 1.5 | g |
| meglumine | 5.7 | g |
| glycerin | 0.819 | g |
| hydrochloric acid | q.s. (pH 8.5) | |
| purified water | q.s. | |
| Total | 100 | mL |

According to each amount defined in the above table, to an appropriate volume of purified water with stirring were added rebamipide, boric acid, meglumine, and glycerin. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Comparative Example 3

| rebamipide | 2 | g |
|---|---|---|
| polyvinylpyrrolidone K17 | 3 | g |
| boric acid | 1.2 | g |
| meglumine | 4.8 | g |
| glycerin | 1.05 | g |
| hydrochloric acid | q.s. (pH 8.5) | |
| purified water | q.s. | |
| Total | 100 | mL |

According to each amount defined in the above table, to an appropriate volume of purified water with stirring were added rebamipide, polyvinylpyrrolidone K17, boric acid, meglumine, and glycerin. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 13

| rebamipide | 2 | g |
|---|---|---|
| polyvinylpyrrolidone K17 | 3 | g |
| boric acid | 1.2 | g |
| meglumine | 4.8 | g |
| glycerin | 1.05 | g |
| hydrochloric acid | q.s. (pH 8.5) | |
| zinc chloride | 2.09 μg (as 1 μg of zinc) | |
| purified water | q.s. | |
| Total | 100 | mL |

According to each amount defined in the above table, to an appropriate volume of purified water with stirring were added rebamipide, polyvinylpyrrolidone K17, boric acid, meglumine, glycerin, and zinc chloride. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 14

A pharmaceutical composition which was a colorless to light yellow clear solution was prepared according to the procedure described in Example 13 except that 4.17 μg of zinc chloride (as 2 μg of zinc) was employed.

### Example 15

A pharmaceutical composition which was a colorless to light yellow clear solution was prepared according to the procedure described in Example 13 except that 6.26 μg of zinc chloride (as 3 μg of zinc) was employed.

### Example 16

A pharmaceutical composition which was a colorless to light yellow clear solution was prepared according to the procedure described in Example 13 except that 8.34 μg of zinc chloride (as 4 μg of zinc) was employed.

### Comparative Example 4

| rebamipide | 2 | g |
|---|---|---|
| polyvinylpyrrolidone K17 | 3 | g |
| boric acid | 1.0 | g |
| meglumine | 4.2 | g |
| glycerin | 1.2 | g |
| hydrochloric acid | q.s. (pH 8.5) | |
| purified water | q.s. | |
| Total | 100 | mL |

According to each amount defined in the above table, to an appropriate volume of purified water with stirring were added rebamipide, polyvinylpyrrolidone K17, boric acid, meglumine, and glycerin. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 17

| rebamipide | 2 | g |
|---|---|---|
| polyvinylpyrrolidone K17 | 3 | g |
| boric acid | 1.0 | g |
| meglumine | 4.2 | g |
| glycerin | 1.2 | g |
| hydrochloric acid | q.s. (pH 8.5) | |
| zinc chloride | 2.09 μg (as 1 μg of zinc) | |
| purified water | q.s. | |
| Total | 100 | mL |

According to each amount defined in the above table, to an appropriate volume of purified water with stirring were added rebamipide, polyvinylpyrrolidone K17, boric acid, meglumine, glycerin, and zinc chloride. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 18

A pharmaceutical composition which was a colorless to light yellow clear solution was prepared according to the procedure described in Example 17 except that 4.17 μg of zinc chloride (as 2 μg of zinc) was employed.

### Example 19

A pharmaceutical composition which was a colorless to light yellow clear solution was prepared according to the procedure described in Example 17 except that 6.26 μg of zinc chloride (as 3 μg of zinc) was employed.

### Example 20

A pharmaceutical composition which was a colorless to light yellow clear solution was prepared according to the procedure described in Example 17 except that 8.34 μg of zinc chloride (as 4 μg of zinc) was employed.

### Comparative Example 5

| rebamipide | 2 | g |
|---|---|---|
| polyvinylpyrrolidone K17 | 3 | g |
| boric acid | 0.5 | g |
| meglumine | 2.7 | g |
| glycerin | 1.54 | g |
| hydrochloric acid | q.s. (pH 8.5) | |
| purified water | q.s. | |
| Total | 100 | mL |

According to each amount defined in the above table, to an appropriate volume of purified water with stirring were added rebamipide, polyvinylpyrrolidone K17, boric acid, meglumine, and glycerin. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 21

| rebamipide | 2 | g |
|---|---|---|
| polyvinylpyrrolidone K17 | 3 | g |
| boric acid | 0.5 | g |
| meglumine | 2.7 | g |
| glycerin | 1.54 | g |
| hydrochloric acid | q.s. (pH 8.5) | |
| zinc chloride | 2.09 μg (as 1 μg of zinc) | |
| purified water | q.s. | |
| Total | 100 | mL |

According to each amount defined in the above table, to an appropriate volume of purified water with stirring were added rebamipide, polyvinylpyrrolidone K17, boric acid, meglumine, glycerin, and zinc chloride. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 22

A pharmaceutical composition which was a colorless to light yellow clear solution was prepared according to the procedure described in Example 21 except that 4.17 μg of zinc chloride (as 2 μg of zinc) was employed.

### Example 23

A pharmaceutical composition which was a colorless to light yellow clear solution was prepared according to the procedure described in Example 21 except that 6.26 μg of zinc chloride (as 3 μg of zinc) was employed.

### Example 24

A pharmaceutical composition which was a colorless to light yellow clear solution was prepared according to the procedure described in Example 21 except that 8.34 μg of zinc chloride (as 4 μg of zinc) was employed.

### Comparative Example 6

| rebamipide | 2 | g |
|---|---|---|
| polyvinylpyrrolidone K17 | 3 | g |
| boric acid | 0.2 | g |
| meglumine | 1.9 | g |
| glycerin | 1.72 | g |
| hydrochloric acid | q.s. (pH 8.5) | |
| purified water | q.s. | |
| Total | 100 | mL |

According to each amount defined in the above table, to an appropriate volume of purified water with stirring were added rebamipide, polyvinylpyrrolidone K17, boric acid, meglumine, and glycerin. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Comparative Example 7

| rebamipide | 2 | g |
|---|---|---|
| polyvinylpyrrolidone K17 | 3 | g |
| Phosphoric acid | 0.0577 | g |
| meglumine | 1.3 | g |
| glycerin | 1.55 | g |
| hydrochloric acid | q.s. (pH 8.5) | |
| purified water | q.s. | |
| Total | 100 | mL |

According to each amount defined in the above table, to an appropriate volume of purified water with stirring were added rebamipide, polyvinylpyrrolidone K17, phosphoric acid, meglumine, and glycerin. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 25

| rebamipide | 2 | g |
|---|---|---|
| polyvinylpyrrolidone K17 | 3 | g |
| boric acid | 0.5 | g |
| meglumine | 2.7 | g |
| glycerin | 1.41 | g |
| hydrochloric acid | q.s. (pH 8.5) | |
| zinc chloride | 2.09 μg (as 1 μg of zinc) | |
| purified water | q.s. | |
| Total | 100 | mL |

According to each amount defined in the above table, to an appropriate volume of purified water with stirring were added rebamipide, polyvinylpyrrolidone K17, boric acid, meglumine, glycerin, and zinc chloride. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Example 26

A pharmaceutical composition which was a colorless to light yellow clear solution was prepared according to the procedure described in Example 25 except that 4.17 μg of zinc chloride (as 2 μg of zinc) was employed.

### Example 27

A pharmaceutical composition which was a colorless to light yellow clear solution was prepared according to the procedure described in Example 25 except that 6.26 μg of zinc chloride (as 3 μg of zinc) was employed.

### Example 28

A pharmaceutical composition which was a colorless to light yellow clear solution was prepared according to the procedure described in Example 25 except that 8.34 μg of zinc chloride (as 4 μg of zinc) was employed.

### Example 29

A pharmaceutical composition which was a colorless to light yellow clear solution was prepared according to the procedure described in Example 25 except that 10.4 μg of zinc chloride (as 5 μg of zinc) was employed.

### Example 30

A pharmaceutical composition which was a colorless to light yellow clear solution was prepared according to the procedure described in Example 25 except that 20.9 μg of zinc chloride (as 10 μg of zinc) was employed.

### Comparative Example 8

| rebamipide | 2 | g |
|---|---|---|
| boric acid | 0.5 | g |
| meglumine | 2.7 | g |
| glycerin | 1.41 | g |
| hydrochloric acid | q.s. (pH 8.5) | |
| zinc chloride | 2.09 μg (as 1 μg of zinc) | |
| purified water | q.s. | |
| Total | 100 | mL |

According to each amount defined in the above table, to an appropriate volume of purified water with stirring were added rebamipide, boric acid, meglumine, glycerin, and zinc chloride. The mixture continued to be stirred with a magnetic stirrer to dissolve, and then the pH thereof was adjusted with hydrochloric acid as shown in the above table. The resulting solution was aseptically filtrated with a 0.2 μm filter to give the desired pharmaceutical composition which was a colorless to light yellow clear solution.

### Comparative Example 9

A pharmaceutical composition which was a colorless to light yellow clear solution was prepared according to the procedure described in Comparative Example 8 except that 4.17 μg of zinc chloride (as 2 μg of zinc) was employed.

### Comparative Example 10

A pharmaceutical composition which was a colorless to light yellow clear solution was prepared according to the procedure described in Comparative Example 8 except that 6.26 μg of zinc chloride (as 3 μg of zinc) was employed.

### Comparative Example 11

A pharmaceutical composition which was a colorless to light yellow clear solution was prepared according to the procedure described in Comparative Example 8 except that 8.34 μg of zinc chloride (as 4 μg of zinc) was employed.

### Comparative Example 12

A pharmaceutical composition which was a colorless to light yellow clear solution was prepared according to the procedure described in Comparative Example 8 except that 10.4 μg of zinc chloride (as 5 μg of zinc) was employed.

### Comparative Example 13

A pharmaceutical composition which was a colorless to light yellow clear solution was prepared according to the procedure described in Comparative Example 8 except that 20.9 μg of zinc chloride (as 10 μg of zinc) was employed.

### Preservation Test

### (Bacterial Strain)

The following bacterial strains were used as inoculums. Bacteria: Escherichia coli ATCC 8739, Pseudomonas aeruginosa ATCC 9027, Staphylococcus aureus ATCC 6538 Yeast and mold: Candida albicans ATCC 10231, Aspergillus brasiliensis ATCC 16404

### (Methods)

The test samples were prepared by aseptically adding each inoculum to the pharmaceutical compositions of Examples and Comparative Examples to obtain each final concentration of 10⁵ to 10⁶ cfu/mL followed by mixing. These samples were stored at 20 to 25° C. under light-shielded condition. 1 mL of an aliquot was collected from each sample on Days 0, 7, 14, and 28, and the viable counts were determined. For determining the viable count of bacteria, 1 mL of a dilution obtained by repeating 10-fold dilution of the aliquot with saline was loaded to a petri dish, 15 to 25 mL of SCDLP agar medium was added thereto, and the viable count was determined after incubation for 5 days at 30 to 35° C. A petri dish whose number of colonies is 300 or less colonies was selected and then the number was multiplied by the dilution ratio to obtain the viable bacteria count. For determining the viable count of yeasts and molds, 1 mL of a dilution obtained by repeating 10-fold dilution of the aliquot with saline was loaded to a petri dish, 15 to 25 mL of GPLP agar medium was added thereto, and the viable count was determined after incubation for 5 days at 20 to 25° C. A petri dish whose number of colonies is 300 or less colonies was selected and then the number was multiplied by the dilution ratio to obtain the viable bacteria count. Survival rates were calculated as a percentage based on the number of colonies at the beginning of the test.

### (Results)

The formulae of Examples 1 to 30 and Comparative Examples 1 to 13, and the results of the preservation test using Staphylococcus aureus are summarized in Tables 1 to 4. Table 5 shows the result of the preservation test of Example 23 using each bacterial strain described above.

The results in Tables 1 to 3 show that a zinc compound in the pharmaceutical compositions contributes to their antibacterial activity, depending on the concentration of zinc.

According to the result in Table 4, the pharmaceutical compositions comprising polyvinylpyrrolidone (Examples 25 to 30) exhibit more potent antibacterial activity than Comparative Examples 8 to 13 which comprise no polyvinylpyrrolidone.

TABLE 1
Bacterial Strain: Staphylococcus aureus ATCC 6538

| | Formulae (/100 mL) | | | | | | | | | |
|---|---|---|---|---|---|---|---|---|---|---|
| | | | | | | zinc chloride | zinc sulfate | | | |
| | | PVP | boric | | | (weight of | (weight of | hydrochloric | Survival rates | |
| | rebamipide | K17 | acid | meglumine | glycerin | zinc) | zinc) | acid | beginning | 7 days later |
| Comparative | 2 g | 3 g | 1.5 g | 5.7 g | 0.819 g | — | — | q.s. (pH 8.5) | 100% | 27.92% |
| Example 1 | | | | | | | | | | |
| Example 1 | 2 g | 3 g | 1.5 g | 5.7 g | 0.819 g | 1 μg | — | q.s. (pH 8.5) | 100% | 13.35% |
| Example 2 | 2 g | 3 g | 1.5 g | 5.7 g | 0.819 g | 5 μg | — | q.s. (pH 8.5) | 100% | 1.74% |
| Exsmple 3 | 2 g | 3 g | 1.5 g | 5.7 g | 0.819 g | 10 μg | — | q.s. (pH 8.5) | 100% | 0.54% |
| Example 4 | 2 g | 3 g | 1.5 g | 5.7 g | 0.819 g | 20 μg | — | q.s. (pH 8.5) | 100% | 0.44% |
| Example 5 | 2 g | 3 g | 1.5 g | 5.7 g | 0.819 g | 50 μg | — | q.s. (pH 8.5) | 100% | 0.11% |
| Example 6 | 2 g | 3 g | 1.5 g | 5.7 g | 0.819 g | — | 1 μg | q.s. (pH 8.5) | 100% | 13.40% |
| Example 7 | 2 g | 3 g | 1.5 g | 5.7 g | 0.819 g | — | 5 μg | q.s. (pH 8.5) | 100% | 1.62% |
| Example 8 | 2 g | 3 g | 1.5 g | 5.7 g | 0.819 g | — | 10 μg | q.s. (pH 8.5) | 100% | 0.60% |
| Example 9 | 2 g | 3 g | 1.5 g | 5.7 g | 0.819 g | — | 20 μg | q.s. (pH 8.5) | 100% | 0.36% |
| Example 10 | 2 g | 3 g | 1.5 g | 5.7 g | 0.819 g | — | 50 μg | q.s. (pH 8.5) | 100% | 0.17% |

PVP: polyvinylpyrrolidone

TABLE 2
Bacterial Strain: Staphylococcus aureus ATCC 6538

| | Formulae (/100 mL) | | | | | | | | |
|---|---|---|---|---|---|---|---|---|---|
| | | | | | | zinc chloride | | | |
| | | PVP | boric | | | (weight of | hydrochloric | Survival rates | |
| | rebamipide | K17 | acid | meglumine | glycerin | zinc) | acid | beginning | 7 days later |
| Comparative | 2 g | — | 1.5 g | 5.7 g | 0.819 g | — | q.s. (pH 8.5) | 100% | 36.44% |
| Example 2 | | | | | | | | | |
| Example 11 | 2 g | — | 1.5 g | 5.7 g | 0.819 g | 20 μg | q.s. (pH 8.5) | 100% | 0.44% |
| Example 12 | 2 g | — | 1.5 g | 5.7 g | 0.819 g | 50 μg | q.s. (pH 8.5) | 100% | 0.28% |

PVP: polyvinylpyrrolidone

TABLE 3
Bacterial Strain: Staphylococcus aureus ATCC 6538

| | Formulae (/100 mL) | | | | | | | | | |
|---|---|---|---|---|---|---|---|---|---|---|
| | | PVP | boric | | | phosphoric | zinc chloride | hydrochloric | Survival rates | |
| | rebamipide | K17 | acid | meglumine | glycerin | acid | (weight of zinc) | acid | beginning | 7 days later |
| Comparative | 2 g | 3 g | 1.5 g | 5.7 g | 0.819 g | — | — | q.s. (pH 8.5) | 100% | 27.92% |
| Example 1 | | | | | | | | | | |
| Comparative | 2 g | 3 g | 1.2 g | 4.8 g | 1.05 g | — | — | q.s. (pH 8.5) | 100% | 27.50% |
| Example 3 | | | | | | | | | | |
| Example 13 | 2 g | 3 g | 1.2 g | 4.8 g | 1.05 g | — | 1 μg | q.s. (pH 8.5) | 100% | 9.13% |
| Example 14 | 2 g | 3 g | 1.2 g | 4.8 g | 1.05 g | — | 2 μg | q.s. (pH 8.5) | 100% | 3.05% |
| Example 15 | 2 g | 3 g | 1.2 g | 4.8 g | 1.05 g | — | 3 μg | q.s. (pH 8.5) | 100% | 2.30% |
| Example 16 | 2 g | 3 g | 1.2 g | 4.8 g | 1.05 g | — | 4 μg | q.s. (pH 8.5) | 100% | 1.16% |
| Comparative | 2 g | 3 g | 1.0 g | 4.2 g | 1.2 g | — | — | q.s. (pH 8.5) | 100% | 18.06% |
| Example 4 | | | | | | | | | | |
| Example 17 | 2 g | 3 g | 1.0 g | 4.2 g | 1.2 g | — | 1 μg | q.s. (pH 8.5) | 100% | 11.10% |
| Example 18 | 2 g | 3 g | 1.0 g | 4.2 g | 1.2 g | — | 2 μg | q.s. (pH 8.5) | 100% | 2.67% |
| Example 19 | 2 g | 3 g | 1.0 g | 4.2 g | 1.2 g | — | 3 μg | q.s. (pH 8.5) | 100% | 1.76% |
| Example 20 | 2 g | 3 g | 1.0 g | 4.2 g | 1.2 g | — | 4 μg | q.s. (pH 8.5) | 100% | 0.72% |
| Comparative | 2 g | 3 g | 0.5 g | 2.7 g | 1.54 g | — | — | q.s. (pH 8.5) | 100% | 10.94% |
| Example 5 | | | | | | | | | | |
| Example 21 | 2 g | 3 g | 0.5 g | 2.7 g | 1.54 g | — | 1 μg | q.s. (pH 8.5) | 100% | 1.61% |
| Example 22 | 2 g | 3 g | 0.5 g | 2.7 g | 1.54 g | — | 2 μg | q.s. (pH 8.5) | 100% | 0.27% |
| Example 23 | 2 g | 3 g | 0.5 g | 2.7 g | 1.54 g | — | 3 μg | q.s. (pH 8.5) | 100% | 0.13% |
| Example 24 | 2 g | 3 g | 0.5 g | 2.7 g | 1.54 g | — | 4 μg | q.s. (pH 8.5) | 100% | 0.21% |
| Comparative | 2 g | 3 g | 0.2 g | 1.9 g | 1.72 g | — | — | q.s. (pH 8.5) | 100% | 10.56% |
| Example 6 | | | | | | | | | | |
| Comparative | 2 g | 3 g | — | 1.3 g | 1.55 g | 0.0577 g | — | q.s. (pH 8.5) | 100% | 4.78% |
| Example 7 | | | | | | | | | | |

PVP: polyvinylpyrrolidone

TABLE 4
Bacterial Strain: Staphylococcus aureus ATCC 6538

| | Formulae (/100 mL) | | | | | | | | |
|---|---|---|---|---|---|---|---|---|---|
| | | | | | | zinc chloride | | | |
| | | PVP | boric | | | (weight of | hydrochloric | Survival rates | |
| | rebamipide | K17 | acid | meglumine | glycerin | zinc) | acid | beginning | 7 days later |
| Example 25 | 2 g | 3 g | 0.5 g | 2.7 g | 1.41 g | 1 μg | q.s. (pH 8.5) | 100% | 2.17% |
| Example 26 | 2 g | 3 g | 0.5 g | 2.7 g | 1.41 g | 2 μg | q.s. (pH 8.5) | 100% | 0.26% |
| Example 27 | 2 g | 3 g | 0.5 g | 2.7 g | 1.41 g | 3 μg | q.s. (pH 8.5) | 100% | 0.14% |
| Example 28 | 2 g | 3 g | 0.5 g | 2.7 g | 1.41 g | 4 μg | q.s. (pH 8.5) | 100% | 0.12% |
| Example 29 | 2 g | 3 g | 0.5 g | 2.7 g | 1.41 g | 5 μg | q.s. (pH 8.5) | 100% | 0.05% |
| Example 30 | 2 g | 3 g | 0.5 g | 2.7 g | 1.41 g | 10 μg | q.s. (pH 8.5) | 100% | 0.03% |
| Comparative | 2 g | — | 0.5 g | 2.7 g | 1.41 g | 1 μg | q.s. (pH 8.5) | 100% | 4.29% |
| Example 8 | | | | | | | | | |
| Comparative | 2 g | — | 0.5 g | 2.7 g | 1.41 g | 2 μg | q.s. (pH 8.5) | 100% | 1.45% |
| Example 9 | | | | | | | | | |
| Comparative | 2 g | — | 0.5 g | 2.7 g | 1.41 g | 3 μg | q.s. (pH 8.5) | 100% | 0.69% |
| Example 10 | | | | | | | | | |
| Comparative | 2 g | — | 0.5 g | 2.7 g | 1.41 g | 4 μg | q.s. (pH 8.5) | 100% | 0.54% |
| Example 11 | | | | | | | | | |
| Comparative | 2 g | — | 0.5 g | 2.7 g | 1.41 g | 5 μg | q.s. (pH 8.5) | 100% | 0.45% |
| Example 12 | | | | | | | | | |
| Comparative | 2 g | — | 0.5 g | 2.7 g | 1.41 g | 10 μg | q.s. (pH 8.5) | 100% | 0.18% |
| Example 13 | | | | | | | | | |

PVP: polyvinylpyrrolidone

TABLE 5

| | Formulae (/100 mL) | | | | | | | | | | |
|---|---|---|---|---|---|---|---|---|---|---|---|
| | | | | | | zinc | | | | | |
| | | | | | | chloride | | | assessment*⁾ | | |
| | | PVP | boric | | | (weight | hydrochloric | | 7 days | 14 days | 28 days |
| | rebamipide | K17 | acid | meglumine | glycerin | of zinc) | acid | Bacterial Strain | later | later | later |
| Example | 2 g | 3 g | 0.5 g | 2.7 g | 1.54 g | 3 μg | q.s. (pH 8.5) | Escherichia coli ATCC 8739 | accept | accept | accept |
| 23 | | | | | | | | Pseudomonas aeruginosa ATCC 9027 | accept | accept | accept |
| | | | | | | | | Staphylococcus aureus ATCC 6538 | accept | accept | accept |
| | | | | | | | | Candida albicans ATCC 10231 | accept | accept | accept |
| | | | | | | | | Aspergillus brasiliensis ATCC 16404 | accept | accept | accept |

PVP: polyvinylpyrrolidone
*⁾Criterion
Bacteria (Escherichia coli, Pseudomonas aeruginosa, Staphylococcus aureus)
7 days later: When it is 10% or less per the beginning, it is assessed as “accept”.
14 days later: When it is 0.1% or less per the beginning, it is assessed as “accept”.
28 days later: When it is equal or less than that of 14 days later, it is assessed as “accept”.
Yeast and mold (Candida albicans, Aspergillus brasiliensis)
7 days later, 14 days later, 28 days later: When it is equal or less than that of the beginning, it is assessed as “accept”.

### Solubility of a Zinc Compound in a Rebamipide-Containing Eye Drop

A composition containing rebamipide was prepared according to the following procedure, and the solubility of the zinc compound in the compositions stored under various conditions was calculated.

### (Preparation of Composition)

2% rebamipide compositions were prepared according to the formula shown below. Each composition contains 5 μg/100 mL or 10 μg/100 mL zinc.

| rebamipide | 2 | g |
|---|---|---|
| polyvinylpyrrolidone K17 | 3 | g |
| boric acid | 0.5 | g |
| meglumine | 2.7 | g |
| glycerin | 1.41 | g |
| hydrochloric acid | q.s. (pH 8.5) | |
| zinc chloride | 10.4 μg (as 5 μg of zinc), or | |
| | 20.9 μg (as 10 μg of zinc) | |
| purified water | q.s. | |
| Total | 100 | mL |

### (Storage Condition)

Each composition was stirred using a magnetic stirrer at 25° C. for 15 days, and precipitation was confirmed. A part of the suspension was taken out and filtered. The concentration of zinc in the filtrate was determined.

Stirring with a magnetic stirrer was continued at 25° C. for additional 8 days (23 days in total). A part of the suspension was taken out and filtered. The concentration of zinc in the filtrate was determined.

The suspension was moved to a refrigerator (5° C.), and stirred with a magnetic stirrer at 5° C. for 53 days (76 days in total). A part of the suspension was taken out and filtered. The concentration of zinc in the filtrate was determined.

### (Results)

The result is shown in the following table. The concentration of zinc in the composition reached a plateau during storage. The solubility of zinc in the rebamipide-containing composition was estimated to be about 3.5 μg/100 mL based on the plateau concentration.

Measurement of zinc concentration solubilized
in 2% rebamipide-containing formulation

| | | zinc concentration (μg/100 mL) | | |
|---|---|---|---|---|
| | | stirred at | stirred at | stirred at |
| | | 25° C., | 25° C., | 5° C., Day 53 |
| Composition | | Day 15 | Day 23 | (76 days total) |
| 2% | zinc added: | 3.72 | 3.56 | 3.47 |
| formulation | 5 μg/100 mL | | | |
| | zinc added: | 6.36 | 6.15 | 3.61 |
| | 10 μg/100 mL | | | |

Reference for the measurement of zinc concentration: Clinica Chimica Acta, 120 (1982) 127-135

1. A pharmaceutical composition comprising (1) rebamipide, (2) a solubilizing agent, (3) an amino sugar, (4) a buffer and (5) a zinc compound.

2. The pharmaceutical composition of claim 1 which is an aqueous liquid.

3. The pharmaceutical composition of claim 1 or 2 wherein the concentration of zinc in the zinc compound is 0.000001 to 0.00005% (w/v).

4. The pharmaceutical composition of claim 1 or 2 wherein the concentration of zinc in the zinc compound is 0.000001 to 0.000004% (w/v).

5. The pharmaceutical composition of any one of claims 1 to 4 wherein the concentrations of rebamipide, the solubilizing agent, the amino sugar, and the buffer are 1 to 3% (w/v), 2 to 4% (w/v), 1 to 6% (w/v), and 0.05 to 2% (w/), respectively.

6. The pharmaceutical composition of any one of claims 1 to 5 wherein the zinc compound is zinc chloride and/or zinc sulfate.

7. The pharmaceutical composition of any one of claims 1 to 6 wherein the solubilizing agent is polyvinylpyrrolidone.

8. The pharmaceutical composition of any one of claims 1 to 7 wherein the amino sugar is meglumine.

9. The pharmaceutical composition of any one of claims 1 to 8 wherein the buffer is boric acid and/or phosphoric acid.

10. The pharmaceutical composition of any one of claims 1 to 9 further comprising an isotonic agent.

11. The pharmaceutical composition of claim 10 wherein the isotonic agent is glycerin.

12. The pharmaceutical composition of any one of claims 1 to 11 wherein pH is in a range of 7 to 9.

13. The pharmaceutical composition of any one of claims 1 to 12 wherein the pharmaceutical composition is an ophthalmic pharmaceutical composition.

14. Use of the pharmaceutical composition of any one of claims 1 to 13 for treating dry eye by topically administering to eyes.
